(19) United States
(12) Patent Application Publication
(10) Pub. No.: US 2019/0388006 A1
(43) Pub. Date: Dec. 26, 2019

(54) NON-INVASIVE SYSTEM AND METHOD FOR BREATH SOUND ANALYSIS

(71) Applicant: Basil Leaf Technologies, LLC, Paoli, PA (US)

(72) Inventors: Basil M. Harris, Paoli, PA (US); Constantine F. Harris, Wyomissing, PA (US); George C. Harris, Ramsey, NJ (US); Edward L. Hepler, Malvern, PA (US)

(73) Assignee: Basil Leaf Technologies, LLC, Paoli, PA (US)

(21) Appl. No.: 16/465,353

(22) PCT Filed: Dec. 8, 2017
(86) PCT No.: PCT/US17/65277

§ 371 Date: May 30, 2019

Related U.S. Application Data
(60) Provisional application No. 62/432,636, filed on Dec. 11, 2016.
(60) Provisional application No. 62/544,472, filed on Aug. 11, 2017.

(51) Int. Cl. A61B 5/08; A61B 7/00; A61B 5/00; A61B 7/04

(52) U.S. Cl. CPC A61B 5/0823; A61B 7/04; A61B 5/7257; A61B 7/003

(57) ABSTRACT

A system for detecting one or more conditions of a subject. The system comprises: a processor; a memory; an intensity mapping component comprising instructions to receive breath sound data for a subject and to determine at least one time-frequency representation of said breath sound data; and a condition identifier component comprising instructions stored in said memory and operable to cause said system to analyze said at least one time-frequency representation to detect one or more conditions as a function of predetermined characteristics of said at least one time-frequency representation, and to store said at least one or more conditions to said memory. Breath sound data may be analyzed to determine whether one or more of a wheeze, a crackle and/or a whooping sound. Detection of wheezes, crackles and/or whoops may be used by an automated diagnostic engine for the purpose of determining a diagnosis.

### CROSS-REFERENCE TO RELATED APPLICATIONS

This application claims the benefit of priority, under 35 U.S.C. § 119(e), of U.S. Provisional Patent Application No. 62/432,636, filed Dec. 11, 2016, and 62/544,472, filed Aug. 11, 2017, the entire disclosures of both of which are hereby incorporated herein by reference.

### FIELD OF THE INVENTION

The present invention relates generally to acquiring and analyzing the breath sounds of a subject and more particularly to a system and method for analyzing the breath sounds to determine if one or more conditions exist that may be signs of disease.

### BACKGROUND

Currently, to analyze the breath sounds of a subject, multiple systems and/or devices are needed, as well as involvement of a trained healthcare professional. This is a very complicated, time-intensive and expensive process. As such, there is a need for a single system that is able to reliably acquire and automatedly analyze breath sounds of a subject to determine if the subject has one or more lung conditions, such as wheezing or crackling. Reliable observations of such conditions may be used to generate a medical diagnosis for the subject.

Further, there is a need to distinguish common cough sounds from diagnosis-specific cough sounds, e.g., to distinguish a whooping cough from a common cough, in support of a diagnosis of pertussis. Pertussis is a bacterial infection caused by bordetella pertussis bacteria, and characterized by a number of different symptoms, such as sneezing, a runny nose, a low-grade fever, and/or diarrhea that are also common symptoms for ailments other than pertussis. However, many people suffering from pertussis develop coughing that often involves coughing spells involving a characteristic “whooping” sound.

Most people in the United States have been vaccinated against pertussis. Despite vaccination, in the U.S. there have been numerous outbreaks of pertussis in recent years. Automated detection of pertussis or whooping cough would be useful.

### SUMMARY

The present invention provides a system for detecting one or more conditions of a subject. The system comprises: a processor; a memory operatively coupled to said processor; an intensity mapping component comprising instructions stored in said memory and operable to cause said system, under control of said processor, to receive breath sound data for a subject from said memory and to determine at least one time-frequency representation of said breath sound data; and a condition identifier component comprising instructions stored in said memory and operable to cause said system, under control of said processor, to analyze said at least one time-frequency representation to detect one or more conditions as a function of predetermined characteristics of said at least one time-frequency representation, and to store said at least one or more conditions to said memory.

In various embodiments, the breath sound data may be analyzed to determine whether one or more of a wheeze, a crackle and/or a whoop sound characteristic of whooping cough has occurred. These analyses may be performed according to predetermined logic, using predetermined parameters. Determination of occurrences of wheezes, crackles and/or whoops may be used by an automated diagnostic engine for the purpose of determining, in conjunction with other objective and subjective data elements, a clinical diagnosis or preliminary determination in support thereof.

### DESCRIPTION OF THE FIGURES

An understanding of the following description will be facilitated by reference to the attached drawings, in which:

FIG. 1a is a block diagram of an exemplary auscultation device in accordance with the present invention;

FIG. 1b illustrates an exemplary housing for the auscultation device;

FIG. 2 is a flow chart illustrating an exemplary method for acquiring audio samples using the auscultation device of FIGS. 1a and 1b;

FIGS. 3 and 4 are block diagrams of a detection system and corresponding components in accordance with the present invention;

FIG. 5 is a flow chart illustrating exemplary methods for generating a time-frequency representation of a subject's breathing using the detection system of FIGS. 3 and 4;

FIG. 6 is a flow chart illustrating exemplary methods for analyzing a time-frequency to identify one or more conditions in a subject's breathing using the Detection System of FIGS. 3 and 4;

FIG. 7a is an exemplary spectrogram generated from a subject's breath sounds;

FIG. 7b is an exemplary spectrogram generated indicating detected wheezes;

FIG. 8 is an exemplary spectrogram generated from the sounds of a subject's breath sounds, indicating detected crackles;

FIG. 9 is an exemplary spectrogram showing normal speech sounds;

FIG. 10 is an exemplary spectrogram showing normal coughing sounds;

FIG. 11 is an exemplary spectrogram cough analysis with subband analysis consistent with an exemplary embodiment of the present invention, showing an absence of whooping sounds;

FIG. 12 is an exemplary spectrogram cough analysis consistent with an exemplary embodiment of the present invention, showing a presence of whooping sounds; and

FIG. 13 is an exemplary spectrogram representing detected whooping sounds, as detected by a system in accordance with the present invention.

### DETAILED DESCRIPTION

The following describes systems and methods for acquiring and analyzing a subject's breath sounds, and for analyzing breath sound data to automatedly process the data to reliably determine occurrences of crackles and wheezes.

One or more signs of disease may be detected for a subject by analyzing a subject's breathing. Signs of disease that may be observed include at least one of wheezing and crackling in the subject's breath sounds. A wheeze is a continuous, coarse, whistling sound produced in the respiratory airways during breathing, and is an indication that at least part of the respiratory tract is narrowed or obstructed, or that airflow velocity within the respiratory tract is heightened. Wheezing is commonly experienced by subjects having a disease, such as asthma, lung cancer, congestive heart failure, or various other types of heart and/or lung diseases. Crackles are popping, clicking, rattling or crackling noises (i.e., rales), that may be occur in one or both lungs with a respiratory disease such as pneumonia, atelectasis, pulmonary fibrosis, acute bronchitis, bronchiectasis, interstitial lung disease, or post-thoracotomy. The presence or absence of one or more of these lung conditions, as well as their locations, can be used to inform a diagnosis determination for a subject.

Further, breath sounds may include coughing sounds. Physiologically, a cough can be described as an expiratory maneuver against a closed glottis, which produces a characteristic sound. Whooping cough involves coughing sounds and whooping sounds. A whooping sound is a different characteristic sound caused by an inspiratory maneuver. A whooping sound is not mere typical inhalation, and it typically follows coughing sounds as the cough sufferer inhales following a series of coughs in a coughing spell.

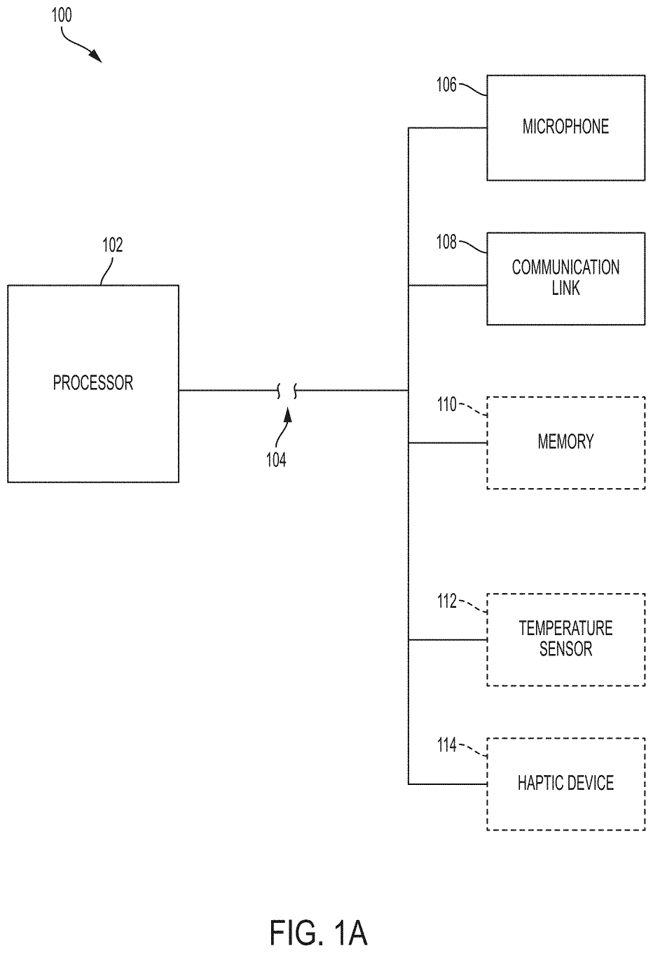

FIG. 1 illustrates a block diagram of an exemplary auscultation device 100 in accordance with the present invention. In the illustrated embodiment, auscultation device 100 comprises a processor 102 coupled to a microphone 106 and a communication link 108 via a communication bus 104. Auscultation device 100 may be coupled to an external computing system via communication link 108, for analysis of data gathered by the auscultation device 100. The external computer system may be any one of a personal computer, tablet, mobile phone, enterprise system or the like.

In various embodiments, processor 102 may also be coupled to an optional memory 110 and/or an optional temperature sensor 112 configured to receive a temperature reading from a subject. Temperature sensor 112 may be swiped across the forehead of the user to acquire a temperature readings. In other embodiments, temperature sensor 112 may be other types of thermometers.

Optionally, in one embodiment, auscultation device 100 further comprises a haptic device 114 configured to provide haptic feedback to a user. Additionally, the external system may include instructions to a subject for positioning the auscultation device 100 relative to anatomical structures, for recording one or more audio samples, etc.

When properly positioned and captured, microphone 106 receives an audio signal corresponding to a subject's breathing (and capturing the subject's breath sounds) and communicates associated audio data to processor 102 via bus 104. In one embodiment, microphone 106 receives and digitizes the audio signal to produce the audio data using an analog-to-digital converter (ADC). Alternatively, the ADC may be an individual component of auscultation device 100, or it may be part of processor 102, or such conversion may occur at an external system. The digital audio data may be communicated to an external computing system via communication link 108, under control of the processor and instructions stored in the memory 110. Communication link 108 may comprise a wireless transceiver or a cable connection to an external computing system. The wireless transceiver may be a Bluetooth transceiver or WLan transceiver. In one embodiment, the wireless transceiver supports Bluetooth low energy or BLE. Audio data acquired by microphone 106 may be streamed to the external computer system via communication link 108. Alternatively, audio data may be stored within memory 110 and communicated to an external computer system at a later point in time.

Processor 102 may be any general purpose microprocessor and an associated system may comprise an internal memory storing instructions and configuration information for the elements of auscultation device 100. In one embodiment, processor 102 communicates instructions stored on an internal memory to microphone 106, instructing microphone 106 to acquire an audio signal. Processor 102 may instruct microphone 106 when to start and when to stop acquiring the audio signal. In one embodiment, an audio sample of a subject's breath sounds is captured for a duration of at least about 10 seconds. Further, audio samples may be acquired at least four different locations on a subject, e.g., locations corresponding to the upper and lower lobes of the right and left lungs. Processor 102 provides instructions to communication link 108 to communicate the digitized audio signal to external computing system for further processing. In one embodiment, processor 102 may apply one or more signal processing techniques to the digitized audio signal before transmission. For example, processor 102 may filter or condition the audio signal before transmission.

In one embodiment, upon start up, auscultation device 100 searches for an external computer system via communication link 108. When an external computer system is detected, a link may be created to the external computer system and the external computer system may begin to send instructions to the auscultation device 100. The instructions may be received by the processor 102 via communication link 108, decoded and then communicated to the corresponding component of auscultation device 100. For example, the external computer system may provide instructions to the auscultation device 100 to begin recording and sending audio signal comprising breath sounds of the subject. Processor 102 may receive and decode the instructions and instruct microphone 104 to begin acquisition of the audio signals. In one embodiment, the instructions provided by the external computer comprise duration settings for each acquisition and/or the number of acquisitions that should be made. Further, the external computer system may provide instructions as to locations relative to anatomical structures as to where on the subject the device should be placed to acquire the audio samples.

Processor 102, microphone 106 and communication link 108 of auscultation device 100 may be housed within a single housing. FIG. 1b shows three different views of a housing for auscultation device 100. The housing may be any shape and/or configuration such that it is able to acquire audio samples corresponding to a subject's breath sounds. One or more indicator lights may be provided to communicate the status of the system. For example, an indicator light may indicate that recording is occurring or has finished (e.g., an indicator light may blink while recording and provide continuous light when finished). Further, an indicator light may be used to provide feedback to a user regarding the amount of interference or noise. If too much noise is detected, an indicator light may provide indication of such to the user. For example, a blinking red light may be used to indicate that there is too much audio signal interference or another issue with the device. In various embodiments, auscultation device 100 may provide feedback regarding the status of the system to external computing system such that it may be displayed to a user.

In certain embodiments, auscultation device 100 is configured to acquire audio signals and also analyze the audio signals to detect one or more conditions as described below. In such embodiments, auscultation device 100 may also comprise at least one of a display device and an input device. The display device may be used to instruct a user how to acquire the audio signals and the input device may be used by a user to interact with the auscultation device 100. In such embodiments, processing system 102 is configured analyze the audio signals to identify lung conditions in addition to providing instructions to the different components of auscultation device 100 and to the subject. In such an embodiment, auscultation device 100 may or may not be coupled to an external computing system.

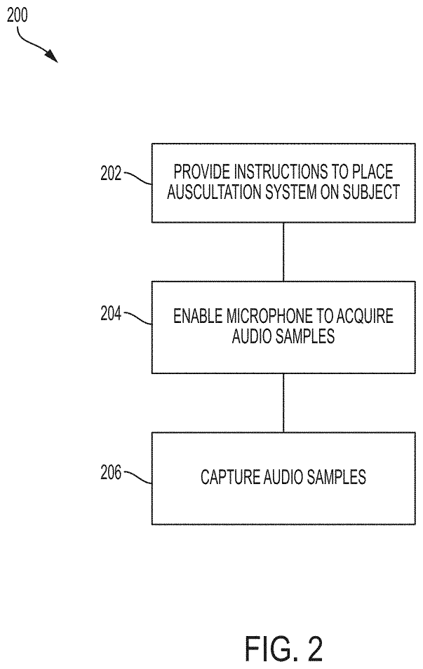

In the exemplary embodiment shown in FIGS. 1a-4, auscultation device 100 is coupled to a separate external computer system, namely detection system 300 shown in FIG. 3, that is configured to process the wheeze and/or crackle information gathered by auscultation device 100. Optionally, detection system 300 may use information from one or more other systems to determine a diagnosis for a subject, or may communication information to an external diagnosis system for determining a diagnosis for a subject. In one embodiment, the diagnosis system is configured to receive and process the breath sound data via communication link 108. In certain embodiments, one or more of the diagnosis system, auscultation device 100 and detection system 300 are incorporated into a single integrated system and device.

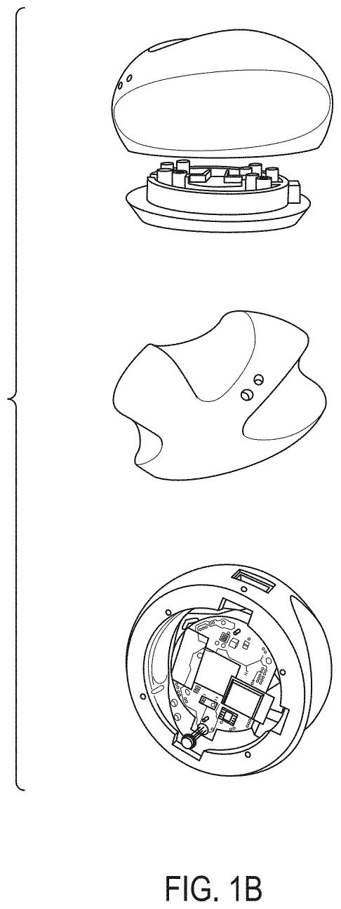

FIG. 2 illustrates an exemplary flow chart 200 for acquiring breath sound data for a subject. While a specific number and order of steps are illustrated, any number of steps may be added or removed and the illustrated steps may be carried out in a different order than that shown. At step 202, instructions are provided to place the auscultation device 100 at one or more locations, e.g., at four different locations on the subject corresponding to upper and lower lobes of each lung. These instructions may be provided by the auscultation device 100, or by an external system, such as the diagnosis system to the Detection System 300. At step 204, the microphone is enabled for recording of an audio signal. In one embodiment, the microphone is enabled after the auscultation device 100 is confirmed to be at the correct location on the subject, e.g., via the subjects input to one of the auscultation device 100, detection system 300 or diagnosis system. An audio sample is acquired using the microphone at step 206. The audio sample is acquired for a predetermined duration of time, and according to a prescribed sample rate, under control of the processor 102. Preferably, the duration is sufficient to allow for multiple inspiration and expiration breathing cycles. In one embodiment, the predetermined duration is at least 10 seconds. Further, the sample rate may be selected to facilitate compression for streaming of audio in real time, to be high enough to meet signal processing requirements, and to be low enough to be compatible with BLE or other communications technologies. By way of example, a sampling rate in the range of about 8 kHz to about 12 kHz may be suitable for this purpose, although any suitable sampling rate may be used. Associated audio data are communicated for processing to identify one or more conditions, e.g., after conversion of the microphone-acquired audio signal to data in digital form.

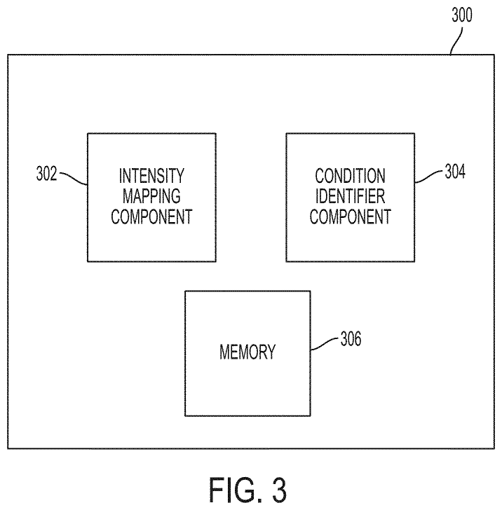

FIG. 3 illustrates a detection system 300 for detecting one or more lung conditions for a subject from the breath sounds of a subject. Detection system 300 comprises intensity mapping component 302, condition identifier component 304 and memory 306. While not shown in FIG. 3, detection system 300 also comprises a processor, such as processor 402, as shown in FIG. 4. Detection system 300 is configured to receive breath sound data associated with an audio signal capturing a subject's breath sounds, process the breath sound data to identify one or more conditions from the breath sound data and store an identification of the detected conditions (and possibly associated detected characteristics) within a memory. Intensity mapping component 302 may be configured to prepare a time-frequency representation based on breath sound data. The time-frequency representation may be prepared by applying a Fast Fourier Transform (FFT) to the breath sound data to transform the data representing the breath sound data into the frequency domain. More specifically, a Short-Time Fourier Transform may be applied to the audio data to generate time-frequency representation for the breath sound data. In one embodiment, a time-frequency determination may be determined for the breath sound data corresponding to each different location on a subject where a breath sound audio signal was captured. Condition identifier component 304 may be configured to analyze each time-frequency representation to detect one or more conditions. Further, the condition identifier component 304 may store the detected one or more conditions in memory 306.

As used herein detection system 300 refers to one or more computing devices configured to detect at least one of a wheeze or a crackle in the breath sounds of a subject. Detection system 300 may comprise any combination of hardware and software aspects of a conventional general purpose computing system, but also further includes instructions for configuring detection system 300 with predetermined rules and/or logic to provide a special-purpose computer system in accordance with the present invention. In one embodiment, detection system 300 comprises computer executable instructions stored on a tangible memory of the system, and the computer executable instructions are executed by a processor within detection system 300 to communicate with a memory, display device, and/or a communication component such as network adapter 410. Detection system 300 may receive breath sound data for a subject via a another device with which it is commutatively coupled or from an element of detection system 300. In one embodiment, detection system 300 receives breath sound data from auscultation device 100. In various embodiments, detection system 300 may be coupled to an internal or external diagnostic system and communicates the identified wheeze or crackle information to the diagnostic system to prepare a diagnosis for the subject. In various embodiments, detection system 300 is configured to generate a diagnosis for a subject at least partially based on the identified wheeze and/or crackle information.

FIG. 4 is a block diagram of an exemplary detection system 300 in accordance with the present invention. Detection system 300 includes conventional computer hardware storing and/or executing specially-configured computer software includes rules and/or logic that configures the hardware as a particular special-purpose machine having various specially-configured functional sub-components that collectively carry out methods in accordance with the present invention. Accordingly, detection system 300 of FIG. 4 includes a general purpose processor 402 and a bus 404 employed to connect and enable communication between the processor 402 and the components of the detection system 300 in accordance with known techniques. The detection system 300 typically includes a user interface adapter 406, which connects the processor 402 via the communication bus 404 to one or more interface devices, such as a keyboard, mouse, and/or other interface devices, which can be any user interface device, such as a touch sensitive screen, digitized entry pad, etc. The bus 404 also connects a display device 408, such as an LCD screen or monitor, to the processor 402 via a display adapter. The bus 404 also connects the processor 402 to memory 306, which can include a hard drive, diskette drive, tape drive, etc. via interface adapter 406. In one embodiment, detection system 300 is also configured to acquire the audio signal, namely the breath sound signal/data, from a user/subject. In such an embodiment, processor 402 is coupled to a microphone, such as microphone 104 via bus 404. Further, processor 402 may be configured to execute the functions discussed in relation to processor 102, to provide the function of device 100.

Detection system 300 may communicate with other computer systems, for example via a communication channel, network adapter 410. The detection system 300 may be associated with such other computer systems in a local area network (LAN) or a wide area network (WAN), and operates as a server in a client/server arrangement with another computer, etc. Such configurations, as well as the appropriate communications hardware and software, are known in the art.

The software of detection system 300 is specially-configured in accordance with the present invention. Accordingly, as shown in FIG. 4, the detection system 300 includes computer-readable, processor-executable instructions 414 stored in the memory 306 for carrying out the methods described herein. For example, memory 306 comprises processor-executable instructions corresponding to one or more of intensity mapping component 302 and condition identifier component 304, as discussed in greater detail below.

Memory 306 may be configured to store data received by or generated from one or more of the components 302, 304. For example, memory 306 may store node breath sound data for a subject received by detection system 300 via network adapter 410. Memory 306 may be further configured to store an identification of at least one of the detected conditions and corresponding characteristics identified by condition identifier component 304.

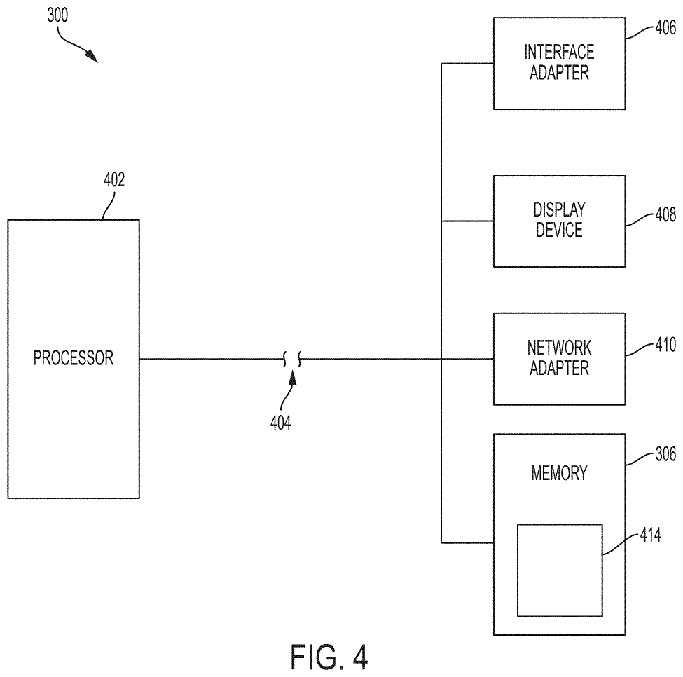

With reference to FIG. 3, intensity mapping component 302 may comprise any combination of software and hardware elements. In one embodiment intensity mapping component 302 comprises computer implemented instructions 414 stored in memory 306 and executable by processor 402. As illustrated in element 502 of the flow chart of FIG. 5, intensity mapping component 302 acquires breath sound data from a memory element, such as memory 306, a communication channel or another component of the system. In one particular embodiment, breath sound data is acquired from upper and lower lobes of each lung of a subject as is discussed above. In one embodiment, intensity mapping component 302 receives instructions to access the breath sound data stored within memory 306 for a subject or a location on a subject. In another embodiment, breath sound data is provided to intensity mapping component 302 via a communication channel. In such an embodiment, intensity mapping component 302 may receive data instructions from processor 402 to access one or more network devices via the communication channel to obtain the breath sound data. Intensity mapping component 302 may be further instructed to access a network device via the communication channel to obtain the breath sound data.

Referring again to FIG. 5, at element 504, intensity mapping component 302 determines a time-frequency representation based on the breath sound data. A time-frequency representation may be determined for breath sound data obtained from each location on a user. In one or more embodiments, the time-frequency representation may be a 3D time-frequency representation or spectrogram. The time-frequency representation may be determined by applying a Fast Fourier Transform (FFT) to the breath sound data to transform the data representing the breath sound data into the frequency domain. More specifically, a Short-Time Fourier Transform may be applied to the audio data to generate the time-frequency representation for the breath sound data. Intensity mapping component 302 may store each time-frequency representation within a memory element, such as memory 306 via bus 404 as shown in element 506. Processing of the representations is described in greater detail below.

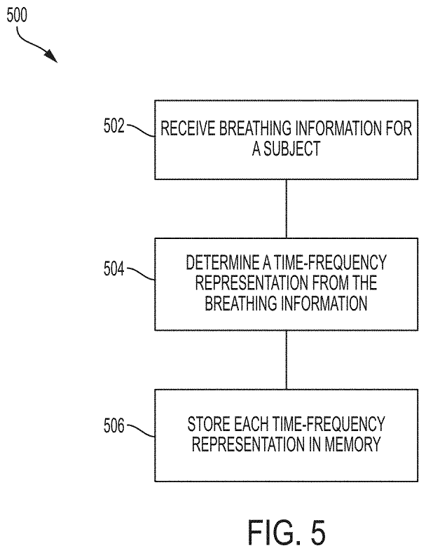

With further reference to FIG. 3, condition identifier component 304 may comprise any combination of software and hardware elements. In one embodiment condition identifier component 304 comprises computer implemented instructions 414 stored in memory 306 executable on processor 402. As illustrated in element 602 of the flow chart of FIG. 6, condition identifier component 304 acquires each time-frequency representation from a memory element, such as memory 306, a communication channel or another component of the system. In one embodiment, condition identifier component 304 receives instructions to access the time-frequency representations stored within memory 306. In another embodiment, time-frequency representations are provided to condition identifier component 304 via a communication channel. In such an embodiment, intensity mapping component 302 may receive data instructions from processor 402 to access one or more network devices via the communication channel to obtain the time-frequency representations. Condition identifier component 304 may be further instructed to access a network device via the communication channel to obtain the time-frequency representations.

Condition identifier component 304 analyzes the time-frequency representation to identify one or more conditions at step 604. The time-frequency representation may be analyzed to determine if one or more of wheezing and crackling are present. As described in greater detail below, condition identifier component 304 analyzes the time-frequency representation to identify one or more of a line of high-intensity frequencies or a band of high-intensity frequencies. A line of high-intensity frequencies that satisfies one or more predetermined thresholds may be deemed to correspond to a wheeze and a band of high-intensity frequencies that satisfies one or more predetermined thresholds may be deemed to correspond to a crackle. In this manner, the condition identifier component 304 can distinguish apparent wheezes/crackles from actual/determined wheezes/crackles to ensure an adequate degree of certainty and reliability in wheeze/crackle detection.

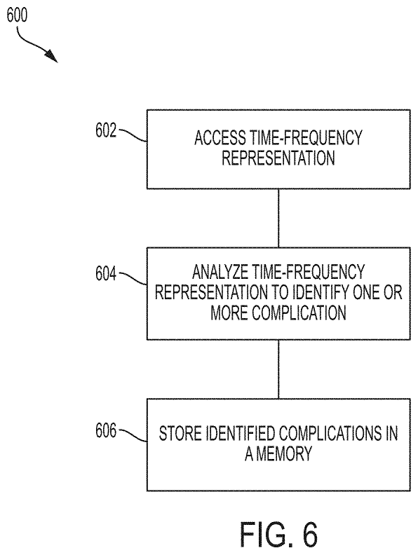

FIG. 7a illustrates a time-frequency representation, or spectrogram, generated from first breath sound data, e.g., breath sound data including patient wheezing. The time-frequency representation depicts the intensity of a frequencies over a period time that the audio signal was acquired. For example, around 1 second there is a high intensity line that slopes up toward 1000 Hz. A similar high intensity line can be found after 2 seconds. In one embodiment, condition identifier component 304 is provided with instructions from processor 402 to apply one or more image analysis and/or data processing techniques to the time-frequency representation to detect the occurrence of a wheeze among the breath sound data. For example, edge detection may be performed by an edge detector on the time-frequency representation to detect a line or ridge of high intensity (e.g., high amplitude) or peak frequencies within the time-frequency representation. Condition identifier component 304 attempts to identify high intensity frequencies (e.g., the frequencies at which high amplitude signals occur), the range of those frequencies, and if those frequencies form a common line (edge or ridge) within the time-frequency representation. An edge or line may be identified as a region of continuous high-amplitude signal for frequencies over a specified frequency range. For example, condition identifier component 304 may attempt to identify a set of continuous high-intensity frequencies between 100 Hz and 800 Hz, however other ranges may be used. A frequency may be determined to be high-intensity (e.g., a peak) when it exceeds a predetermined threshold amplitude. The threshold amplitude may be based on the intensities (e.g., amplitudes) of other adjacent frequencies within the representation. Condition identifier component 304 may also be configured to identify one or more harmonics of each high intensity frequency, and the presence, absence or nature of harmonics may be used to distinguish apparent wheezes from determined wheezes by comparison to predetermined thresholds or rules. Once frequencies forming a common line have been identified, the duration of that line may be determined. Duration may be determined by measuring the amount of time between a first frequency and last frequency forming a line (or ridge). Additionally, the slope of the line may be determined for the frequencies forming the line. Duration and slope may also be used to determine the presence of wheeze, according to predetermined thresholds or rules of the condition identified component. In one embodiment, a wheeze is determined to be an actual determined wheeze if it has at least one of a base frequency in range of 100 hertz to 800 hertz, a duration of at least 100 ms and having a rising pitch where the corresponding slope is relatively flat or increasing. Further, in one embodiment wheezes are characterized as tones having at least two harmonics. In various embodiments, the frequency range used to identify a wheeze may be below 100 hertz or exceed 800 hertz depending on the embodiment. Further, the duration may be less than 100 milliseconds in various embodiments. In one particular embodiment, the slope of a line representing a wheeze is about 2000 Hz/second.

In one or more embodiments, a wheeze may be identified using one or more of the above characteristics, and then confirmed using other characteristics. Confirmation may occur within detection system 300 or within a diagnosis or other external system. In one embodiment, condition identifier component 304 is configured to determine the possibility that a wheeze may be present may depend on the number of characteristic that are identified within the time-frequency representation. For example, condition identifier component 304 may determine that there is a low probability that a wheeze is present when only one characteristic is identified, and a higher probability when multiple characteristics are identified. In various embodiments, the duration of a wheeze may be used to determine the probability that a wheeze exists. An apparent wheeze lasting longer than about 300 ms may be considered a long wheeze and may have a high probability of being an actual wheeze while an apparent wheeze lasting between about 100 ms—about 300 ms may have a low probability of being an actual wheeze. In one embodiment, a long wheeze corresponds to a wheeze of at least 300 ms in duration. Additionally, the length of the wheeze may be used to generate the diagnosis of a subject. The length of the wheeze may refer the longest wheeze identified, the average length of identified wheezes, the shortest wheeze identified, etc. While specific thresholds are discussed above, any suitable predetermined thresholds, rules and logic may be used for wheeze determination and probability determination. Condition identifier component 402 may also determine the number of wheezes that are within the sampling period.

In one or more embodiments, as the number of characteristics identified increases, the probability or confidence that an actual wheeze is present may also increase. Characteristics determined from the breath sound data from each location on a subject may be correlated to determine the probability that a wheeze was present. For example, if a wheeze was present in the information from only one location on a user, the probability of that being an actual wheeze may be lower if a wheeze was identified in the information pertaining to more than one location. In various embodiments, information from different locations on the subject may be correlated to increase the probability that a wheeze exists. Further, as the number of wheezes are identified the probability that wheeze is present increases.

FIG. 7b shows the spectrogram of FIG. 7a with identified wheezes meeting predetermined wheeze-detection criteria. Four separate wheeze occurrences are identified in FIG. 7b, as indicated by elements 702-708. As can be seen, each identified wheeze has high intensity of frequencies between about 800 Hz to 1000 Hz, and form a ridge with an either flat or increasing slope. Further, each identified wheeze has a duration of at least 100 ms and has at least two harmonics. According to predetermined thresholds, rules and/or logic, the apparent wheeze is deemed a determined wheeze if it meets the predetermined wheeze identification criteria.

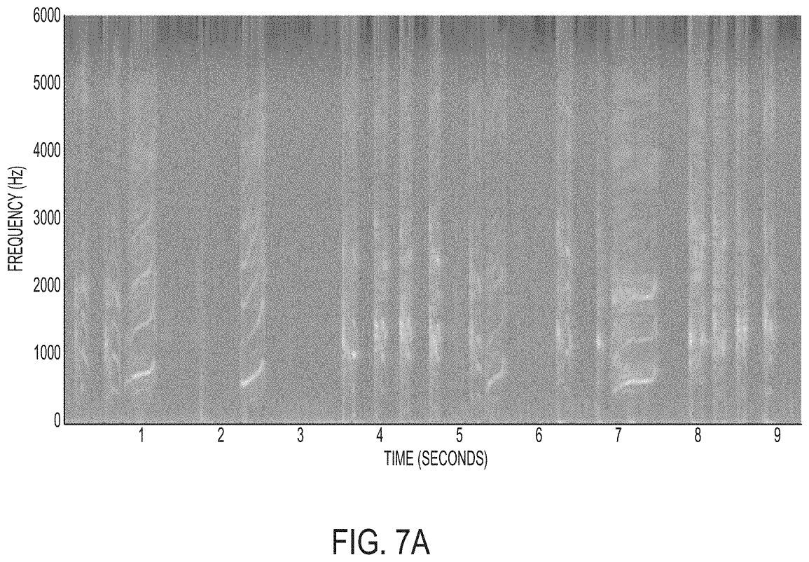

FIG. 8 illustrates an exemplary time-frequency representation, or spectrogram, identifying a plurality of crackles. In one embodiment, condition identifier component 304 analyzes the time-frequency representation to detect characteristics of crackles. For example, condition identifier component 304 may apply one more image processing or other data processing techniques to the time-frequency representation to identify the characteristics of one or more crackles. In one embodiment, condition identifier component 304 is configured to scan the time-frequency representation to determine instances of a large amount (e.g., a relatively large proportion) of spectral energy concentrated in high frequency bands, as this is deemed to be characteristic of an occurrence of a crackle. The highlighted portion of FIG. 8, 802, indicates a portion of the time-frequency representation where this is a large amount of spectral energy concentrated at higher frequencies. The presence of a crackle at a particular time is determined at a time where the proportion of energy in the spectrum above a certain threshold frequency exceeds another predetermined threshold. These thresholds were determined by analyzing audio samples of known breath sound stethoscope samples with crackles. The spectral characteristics of known crackle waveforms were analyzed and the cutoff thresholds for energy distribution and the duration of crackles was determined. In one embodiment, a crackle is determined to exist where the percentage of energy within a predetermined high frequency portion of the spectrum exceeds a predetermined threshold for a predetermined short duration of time, when examining the time-frequency representation of the breath sound signal. In one embodiment, the predetermined short duration of time is a duration of about 10 ms or less. In other embodiments, the duration of the band may exceed 10 ms. By scanning the intensities for each frequency, higher frequencies having higher intensities may be detected. In one embodiment, the total energy contained in these frequencies may be calculated and compared against a predetermined threshold level. In one embodiment, the energy threshold level may be a percentage of energy that exists in the higher frequency bands for a given time period. In one particular embodiment, the time period, or duration, may be 10 ms or less. In other embodiments, the time period may be greater than 10 ms. In one embodiment, a crackle was determined to be present based on a threshold percentage of energy that was concentrated above a cutoff frequency as compared to the total signal energy in the given time period. For example, in one embodiment, if more than 10% of the spectral energy is above a cutoff frequency of 1000 Hz in a given 10 ms time period, then an apparent crackle is deemed to be a determined (actual) crackle. In other embodiments, the threshold percentage of energy may be less than or greater than 10%. Further, in various embodiments, the cutoff frequency may be greater than 1000 Hz or less than 1000 Hz.

In one embodiment, the probability that a crackle exists is determined. The probability may be based upon the number of crackles detected in a sample, the duration of detected crackles and/or the amount of energy that is determined to be in the higher frequencies. As the number of crackles detected and/or the amount of energy increases, the probability or likelihood that a crackle exists increases. Further, as the crackles are found to be closer to the threshold duration, the probability that a crackle exists increases. In various embodiments, characteristics determined from the breath sound data from each location on a subject may be correlated to determine the probability that a crackle was present. For example, if a crackle was present in the information from only one location on a user, the probability of that being an actual crackle may be lower if a crackle was identified in the information pertaining to more than one location. Condition identifier component 402 may also determine the number of crackles over the sampling period. In various embodiments, crackle information from different locations on the subject may be correlated to increase the probability that a crackle exists.

At element 606 of flowchart 600, condition identifier component 402 stores the identified conditions in a memory, such as memory 402. In one embodiment, characteristics identifier 402 not only stores the identified condition, wheeze or crackle, but also the characteristic information related to the wheeze or crackle. For example, condition identifier component 304 may be configured to store one or more of the duration, slope, frequency range and number of harmonics for a wheeze. Further, the condition identifier component 304 may be configured to store a probability score for a wheeze. In another example, condition identifier component 304 may be configured to store one or more of a duration and energy concentration percentage for a crackle. Further, the condition identifier component 304 may be configured to store a likelihood or probability score for a crackle. In one or more embodiments, the characteristics for each wheeze and crackle is correlated with the breath sound data to identify which portion of a lung of a subject the crackle or wheeze occurred in.

In one or embodiments, condition identifier component 304 may communicate via a communication channel, one or more of a detected wheeze and it's characteristics, and a detected crackle and its characteristics, to an internal or external diagnostic system. The diagnostic system may use the wheeze and crackle information to generate a diagnosis for the subject.

In various embodiments, information from other devices may be correlated to the wheeze or crackle information to identify additional characteristics. In one embodiment, detection system 300 correlates information received from an impedance pneumography device with the breath sound data to determine if an identified wheeze or crackle is inspiratory or expiratory, and such inspiratory and expiratory information may be communicated to a diagnostic system, e.g., for the purpose of diagnosis development.

In one embodiment, the device is used to identify whooping cough, e.g., to identify coughs among microphone-recorded breath sounds, and further to analyze a corresponding audio signal to determine whether or not whooping sounds are present, in support of a potential diagnosis of whooping cough or pertussis. In such an embodiment, the functionality of the device 100 may be integrated into a general purpose computing device, such as a personal computer, tablet PC, smartphone, or even a personal assist device such as an Amazon Echo device, that includes a processor 102 coupled to a microphone 106 and a communication link 108 via a communication bus 104, and that is configured to perform the audio signal analyses described herein, or to transmit data to an external computing system configured to perform the audio signal analyses described herein. In one exemplary embodiment the device 100 maybe configured to selectively or continuously capture an audio signal via the microphone 106, and to digitize the audio signal, e.g., using an analog-to-digital (ADC) converter as discussed above.

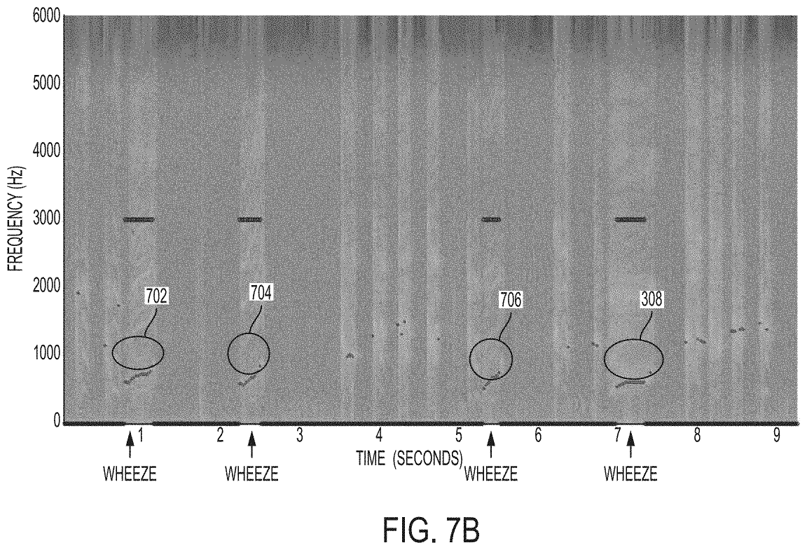

It will be appreciated that such an approach may result in recording of background noise, speech, or other signals other than breath sounds, and in particular cough sounds. FIG. 9 is an exemplary spectrogram (time-frequency representation) showing exemplary normal speech sounds resulting from recording of speech by a microphone 106 of the device 100. The spectrogram may be created by an intensity mapping component 302, as described above. Accordingly, in this embodiment whooping cough detection involves identifying cough sounds from among background noise, speech and other breath sounds that may be captured and/or recorded by the microphone. Detection of one or more cough sounds may then be used to initiate further analysis to determine whether whooping sounds are present, and whether the audio signal is representative of whooping cough.

In an exemplary embodiment, this is performed by analyzing the captured audio signal to identify whether all, or a sufficient number, of the following characteristics are found, as each of these is deemed to be representative of a presence of a cough sound. Such analysis may be performed by the condition identifier component 304, as described above. The sensitivity of the system may be configured by adjusting the number of characteristics that are required within a particularly embodiment of the system to produce a determination that cough sounds are present. By way of example, this may be performed by periodically or successively by processing microphone-captured audio signal in intervals, such as 10-second intervals.

A first characteristic involves finding of a sudden increase in volume over an ambient audio level. This can be determined by an increase in the RMS energy level by a predetermined number of decibels over the ambient sound level. The particular predetermined number used may be chosen empirically after analyzing a database of cough sound audio samples. If the system determines that suspected cough sounds meet predetermined thresholds for sudden volume increase over an ambient audio level, then, the system may determine that cough sounds are present.

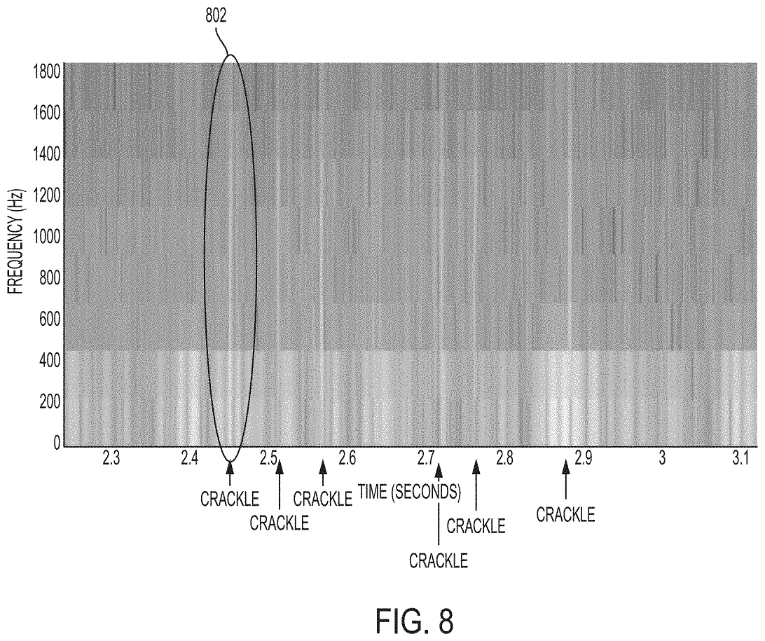

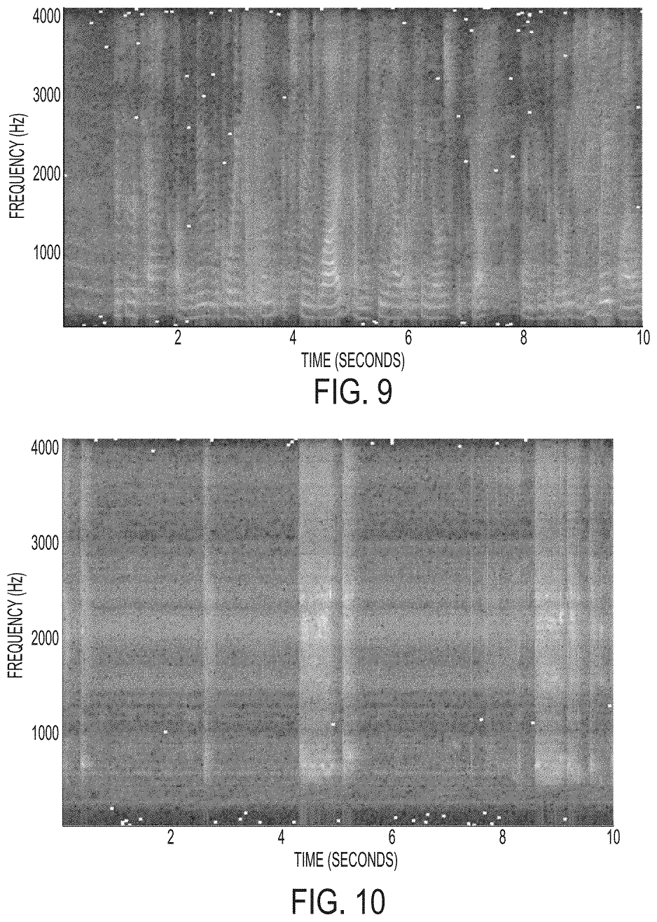

A second characteristic involves a finding of an even energy distribution across frequencies. This can be determined by processing the captured audio signal to transform it to a frequency domain, e.g., using a Fast Fourier Transform, by performing a short time Fourier transform a corresponding spectrogram comprising a three-dimensional time-frequency representation of the signal may be created. FIG. 10 is an exemplary spectrogram showing normal coughing sounds. The spectrogram, and thus the spectral characteristics of the audio signal, may then be analyzed at each of a plurality of time periods. For example, the spectrum may be divided into N linearly spaced subbands of equal width from low frequency to highest frequency. In one exemplary embodiment, five (5) subbands having a width 800 Hz each may be used. The energy distribution across the subbands may then be compared. If the energy is spread out relatively equally across the plurality of subbands, then the system may determine that the captured audio signal includes cough sounds. Various suitable parameters may be used to determine when the energy is spread out relatively equally. FIG. 11 is an exemplary spectrogram cough analysis with subband analysis consistent with an exemplary embodiment of the present invention. This Fig. shows a presence of cough sounds, but also an absence of whooping sounds, as discussed below.

A third characteristic involves a finding that the captured (potentially cough) sounds are characteristically short in duration. This can be determined by analyzing the captured audio signals, identifying the cough portions as portions having sufficiently high (according to predetermined thresholds) volume over ambient audio level, and determining the duration in time of those portions and comparing them to a predetermining duration threshold. The particular volume and time duration thresholds used may be empirically chosen after analyzing a database of cough sound audio samples. In one embodiment, the system may determine a suspected cough sound having a duration of 300 ms to 600 ms to be a cough sound.

A fourth characteristic involves a finding that the captured (potentially cough) sounds are clustered within a short time period. Accordingly, if more than a predetermined number of suspected cough sounds are found within a predetermined time period, then the system may determine that cough sounds are present.

A fifth characteristic involves a finding that he captured (potentially cough) sounds immediately follow a deep inspiration sound. In such an embodiment, the system includes a respiratory phase sensor on an auscultation device in addition to the microphone 106, and is configured to identify deep inspiration sounds as captured by the respiratory phase sensor. In the event that suspected cough sounds are found within a predetermined time duration following a deep inspiration sound, then the system may determine that cough sounds are present.

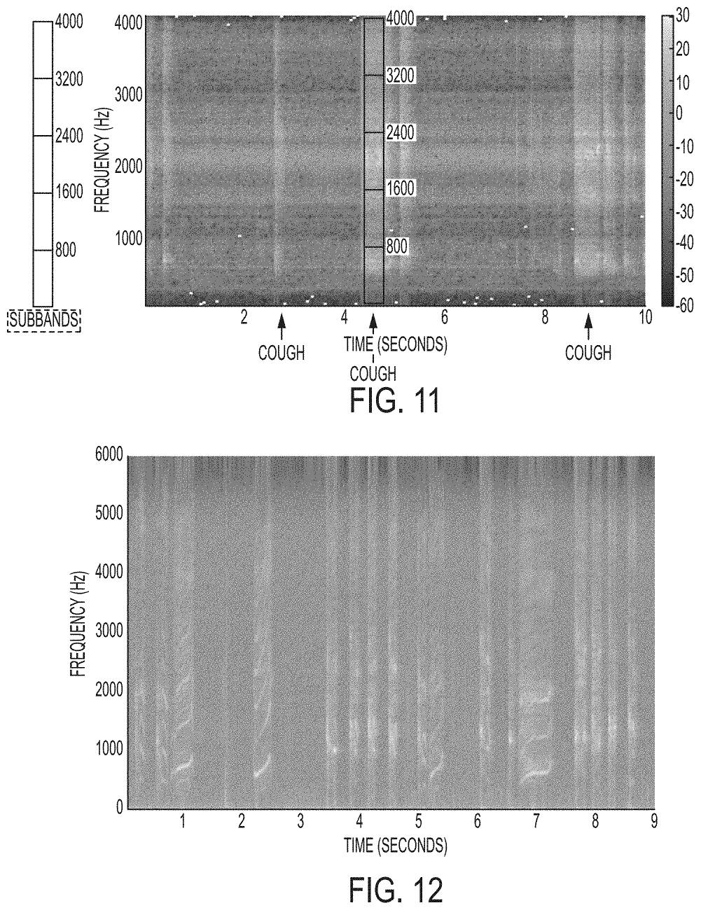

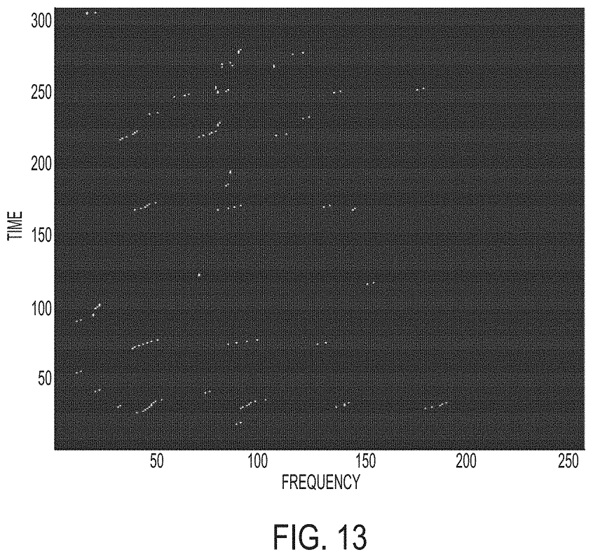

After the system has determined that cough sounds are present in the captured audio signal, it then further analyzes the captured audio signal to determine whether whooping sounds are present. More particularly, the spectrogram (time-frequency image) is further analyzed. FIG. 12 is an exemplary spectrogram of cough sounds, showing a presence of whooping sounds. More particularly, a three-dimensional peak edge detector is used to scan the time-frequency spectrogram image to detect peaks (generally represented by horizontal or diagonal lines, or approximations of lines). FIG. 13 is an exemplary spectrogram representing detected whooping sounds, with an enhanced depiction of edge detection, as detected by a system in accordance with the present invention.

The system may be configured to determine that whooping sounds exist if all, or a predetermined number or combination, of the following characteristics exist.

A first characteristic is a finding of a presence of tones with rising frequencies, multiple harmonics, and a base frequency in a predetermined range, and thus what appears as generally parallel sloping lines in the spectrogram. In one embodiment, the predetermined range is 500 Hz to 1000 Hz, though other frequency ranges may be used for this purpose.

A second characteristic is a finding of tones with a slope, from start to finish, within a predetermined range. In one embodiment, the predetermined range is 0 Hz/second to 2000 Hz/second, though other slope ranges may be used for this purpose.

A third characteristic is a finding of tones with a duration, from start to finish, within a predetermined range. In one embodiment, the predetermined range is greater than 90 milliseconds, though other duration ranges may be used for this purpose.

A determination of an occurrence of a whoop may be performed in automated fashion according to predetermined logic, consistent with the characteristics defined above and/or other suitable parameters, thresholds and/or logic, as will be appreciated by those skilled in the art. For example, these three characteristics could be considered in combination and be combined into a score representing a likelihood regarding the presence of a “whoop”. In either case, the presence or absence of the “whoop” may be recorded and incorporated into a data store that may be used by a computerized diagnostic system for making a diagnostic assessment based on the presence of absences of a “whoop” (e.g., by assigning probabilities to differential diagnoses based on the available data).

Generally, several factors suggest that a whoop is more likely to be present, and may be considered by the system according to predetermined logic to support a determination as to whether or not a “whoop” was detected. For example, if any of the following criteria are met then the presence of a whoop may be considered more likely: detection of a whooping sound immediately following a detected cough sound; a duration of the whooping sound that is determined to be sufficiently long (in duration (e.g., greater than T seconds, where T was determined from analyzing a database of actual whooping cough sounds); and/or the whoop sound is found to occur during a detected inspiration, e.g., by correlating the breathing audio signal with impedance pneumography, to distinguish between inspiration and exhaling, it being recognized that whooping typically occurs during the inspiration phase.

If more than one of these criteria are met then the presence of a whoop sound may be considered to be “very likely”, and may be reflected in the probability, weighting, or conclusion in any fashion desired, according to a predetermined methodology.

In one or embodiments, condition identifier component 304 may communicate via a communication channel its determination of whether or not whooping cough has been detected to an internal or external diagnostic system. The diagnostic system may use the whooping cough detection determination to generate a pertussis diagnosis for the subject.

For example, detection of a characteristic whoop sound is a notable objective measure when performing a clinical assessment. However, when taken alone, the presence or absence of a whoop does not rule-in or rule-out any particular disease. Rather, it is one of many data points, both objective and subjective, that when taken together inform the clinical diagnosis. Other objective measures may include, for example, presence or absence of fever as well as other vital signs (e.g., heart rate, oxygen saturation, respiratory rate). Some subjective data points may include muscle aches or chills. The patient's medical and social history also plays a role in making a diagnosis, for example recent exposures. A suitable diagnostic system may be configured to make a diagnosis according to defined algorithms that may place a weighted value on each individual data point. Thereby, all of the objective and subjective elements contribute in some way in favor of or against a particular clinical diagnosis. Some elements may be more important than others for specific diagnoses but seldom is one data point alone weighted so heavily that it alone can yield a clinical diagnosis. The algorithm may account for different weightings of differed objective and subjective elements as appropriate for making a desired diagnosis.

In various embodiments, information from other devices may be correlated to the whooping cough detection determination to identify additional characteristics. In one embodiment, detection system 300 correlates information received from other sensors and communicates relevant information to a diagnostic system, e.g., for the purpose of diagnosis development.

A computer program product stored on a tangible computer-readable medium for carrying out the methods identified above is provided also. The computer program product comprises computer readable instructions for carrying out the methods described herein. In one embodiment, an exemplary computer program product comprises a tangible computer-readable medium storing a software application providing the functionality described herein.

While certain embodiments according to the invention have been described, the invention is not limited to just the described embodiments. Various changes and/or modifications can be made to any of the described embodiments without departing from the spirit or scope of the invention. Also, various combination of elements, sets, features, and/or aspects of the described embodiments are possible contemplated even if such combinations are not expressly identified herein.

1. A system for detecting one or more conditions of a subject, said system comprising:
a processor;
a memory operatively coupled to said processor;
an intensity mapping component comprising instructions stored in said memory and operable to cause said system, under control of said processor, to receive breath sound data for a subject from said memory and to determine at least one time-frequency representation of said breath sound data; and
a condition identifier component comprising instructions stored in said memory and operable to cause said system, under control of said processor, to analyze said at least one time-frequency representation to detect one or more conditions as a function of predetermined characteristics of said at least one time-frequency representation, and to store said at least one or more conditions to said memory.

2. The system of claim 1, wherein said one or more conditions comprises at least one of at least one wheeze and at least one crackle.

3. The system of claim 1, wherein said intensity mapping component applies at least one of a Fast Fourier Transform and a Short Time Fourier Transform to said breath sound data to said breath sound data to determine said at least one time-frequency representation.

4. The system of claim 1, wherein said breath sound data comprises breath sound data recorded for at least two locations on the subject, and wherein determining said at least one time-frequency representation comprises determining a first time-frequency representation corresponding to breath sound data recorded at a first location of said user and a second time-frequency representation corresponding to breath sound data recorded at a second location of said user.

5. The system of claim 1, wherein analyzing said at least one time-frequency representation to detect said one more conditions comprises detecting a high-intensity frequency ridge within said time-frequency representation.

6. The system of claim 5, further comprising determining a duration of said high-intensity frequency ridge and comparing said duration to a duration threshold to detect said one or more conditions.

7. The system of claim 6, wherein said duration threshold is 100 ms.

8. The system of claim 5, further comprising determining a frequency range of said high-intensity frequency ridge and comparing said frequency to a frequency range threshold to detect said one or more conditions.

9. The system of claim 7, wherein said frequency range threshold is a range of 100 Hz to 800 Hz.

10. The system of claim 5, further comprising determining a slope of said high-intensity frequency ridge end comparing said slope to a slope threshold to detect said one or more conditions.

11. The system of claim 10, wherein said slope threshold is 2000 Hz per second.

12. The system of claim 5, further comprising determining a number of harmonics of frequency in said high-intensity frequency ridge end comparing said number of harmonics to a harmonic threshold to detect said one or more conditions.

13. The system of claim 5, wherein said harmonic threshold is two.

14. The system of claim 1, wherein analyzing said at least one time-frequency representation to detect said one more conditions comprises detecting a concentration of high frequency bands of said time-frequency representation.

15. The system of claim 14, further comprising determining a duration of said concentration of high frequency bands and comparing said duration to a duration threshold to detect said one or more conditions.

16. The system of claim 15, wherein said duration threshold is 10 ms.

17. The system of claim 16, further determining a percentage of energy of said concentration of high frequency bands is above a cutoff frequency and determining said amount of energy to an energy threshold to detect said one or more conditions.

18. The system of claim 17, wherein said energy threshold is 10% and wherein said cutoff frequency is 1000 Hz.

19. A method for detecting one or more conditions of a subject, said method comprising:
acquiring breath sound data corresponding to breathing of said subject;
determining at least one time-frequency representation based on said breath sound data; and
analyzing said at least one time-frequency representation to detect said one more condition.

20. (canceled)

21. The method of claim 19, wherein determining said time-frequency representation based on said breath sound data comprises applying at least one of a Fast Fourier Transform and a Short Time Fourier Transform to said breath sound data.

22. The method of claim 19, wherein analyzing said at least one time-frequency representation to detect said one more conditions comprises detecting a high-intensity frequency ridge within said time-frequency representation.

23-24. (canceled)

25. The method of claim 22, further comprising determining a slope of said high-intensity frequency ridge end comparing said slope to a slope threshold to detect said one or more conditions, wherein said slope threshold is 0 Hz per second.

26. The method of claim 22, further comprising determining a number of harmonics of frequency is said high-intensity frequency ridge end comparing said number of harmonics to a harmonic threshold to detect said one or more conditions, wherein said harmonic threshold is two.

27. The method of claim 19, wherein analyzing said at least one time-frequency representation to detect said one more conditions comprises detecting a concentration of high frequency bands of said time-frequency representation.

28. The method of claim 27, further comprising determining a duration of said concentration of high-frequency bands and comparing said duration to a duration threshold to detect said one or more conditions wherein said duration threshold is 10 ms.

29. The method of claim 27, further determining a percentage of energy of said concentration of high-frequency bands is above a cutoff frequency and determining said amount of energy to an energy threshold to detect said one or more conditions, wherein said energy threshold is 10% and wherein said cutoff frequency is 1000 Hz.

30. (canceled)

31. The method of claim 19, wherein said one or more conditions comprises a whoop, and wherein analyzing said at least one time-frequency representation to detect said whoop comprises determining said time-frequency representation based on said breath sound data comprises:
applying at least one of a Fast Fourier Transform and a Short Time Fourier Transform to said breath sound data;
detecting a high-intensity frequency ridge within said time-frequency representation; and
determining whether a whoop has occurred as a function of an analysis of the breath sound data.

32. The method of claim 31, wherein determining whether a whoop has occurred comprises determining a whoop has occurred when a tone in the breath sound data has a base frequency in the range of 500 Hz to 1000 Hz, a rising frequency over time, and multiple harmonics.

33. The method of claim 31, wherein determining whether a whoop has occurred comprises determining a whoop has occurred for a breath sound that occurred in succession to a detected cough sound.

34. The method of claim 31, wherein determining whether a whoop has occurred comprises determining a whoop has occurred for a breath sound that occurred during an inspiration phase of breathing.

35. The method of claim 31, further comprising determining a duration of said high-intensity frequency ridge and comparing said duration to a duration threshold to detect said whoop, wherein said duration threshold is greater than 90 ms.

36. The method of claim 31, further comprising determining a slope of said high-intensity frequency ridge end comparing said slope to a slope threshold to detect said whoop, wherein said slope threshold is between 0 Hz per second and 2000 Hz per second.

37. An auscultation device comprising:
a microphone configured to acquire breath sounds from a subject;
a communication link configured to couple to an external computing system; and
a processor configured to provide instructions to said microphone to acquire said breath sounds and said communication link to communicate said acquired breath sounds to said external computing system.

38. The auscultation device of claim 37, wherein said acquired breath sounds are streamed to said external computing system via said communication link.

39. The auscultation device of claim 37, wherein said communication link comprises a wireless connection.

40. The auscultation device of claim 37, wherein said microphone, said processor and said communication link are disposed within a common housing.
